SEQUENCE LISTING (4 sequences)

SEQ ID NO: 1 - protein, 60 aa, Drosophila melanogaster
RKRGRQTYTRYQTLELEKEFHFNRYLTRRRRIEIAHALCLTERQIKIWFQNRRMKWKKEN

SEQ ID NO: 2 - protein, 4 aa, Artificial Sequence
  Synthetic construct
IEGR

SEQ ID NO: 3 - protein, 5 aa, Artificial Sequence
  Synthetic construct
DDDDK

SEQ ID NO: 4 - protein, 5 aa, Artificial Sequence
  Synthetic construct
LVPRG

(19) United States
(12) Patent Application Publication
(10) Pub. No.: US 2010/0266592 A1
(43) Pub. Date: Oct. 21, 2010

(54) THERAPEUTIC ANTENNAPEDIA-ANTIBODY MOLECULES AND METHODS OF USE THEREOF

(75) Inventors: Agamemnon A. Epenetos, London (GB); Christina Kousparou, Nicosia (CY)

Correspondence Address: DLA PIPER LLP (US), 4365 EXECUTIVE DRIVE, SUITE 1100, SAN DIEGO, CA, 92121-2133, US

(73) Assignee: Trojan Technologies, Ltd., London (GB)

(21) Appl. No.: 12/760,425

(22) Filed: Apr. 14, 2010

Related U.S. Application Data
(60) Provisional application No. 61/169,166, filed on Apr. 14, 2009.

(51) Int. Cl. A61K 39/395; C07K 16/00; C07H 21/00; C12N 5/10; C12N 5/071; A61K 31/7088; C12N 5/09; A61P 35/00

(52) U.S. Cl. 424/134.1; 530/387.3; 536/23.4; 435/325; 435/366; 514/44 R

(57) ABSTRACT

The present invention is based on the seminal discovery that an antibody or fragment thereof combined with Antennapedia or a fragment thereof, is an effective therapeutic agent. The invention describes the construction of antibody or antibody fragment fused or chemically conjugated with Antennapedia at its carboxyl or its amino terminus (a “cargo-carrier” construct).

### CROSS REFERENCE TO RELATED APPLICATION(S)

This application claims the benefit of priority under 35 U.S.C. §119(e) of U.S. Ser. No. 61/169,166, filed Apr. 14, 2009, the entire content of which is incorporated herein by reference.

### BACKGROUND OF THE INVENTION

1. Field of the Invention

The invention relates generally to delivery of molecules to cells and more specifically to therapeutic antibody-protein conjugates containing Antennapedia (Antp) or a fragment of Antp.

2. Background Information

The gene antennapedia (Antp) encodes a transcriptional factor that has been shown to control antero-posterior morphogenesis in Drosophila embryo. The protein sequence of antennapedia is characterised by the presence of a 60 amino acids motif (homeodomain) that binds to specific DNA target elements. Antennapedia homologues have been found in nearly all multicellular organisms and show a very high degree of amino acid sequence identity. The human and drosophila antennapedia proteins differ in the sequence of the homeodomain only for one conservative amino acid substitution.

It has been observed that antennapedia and its homeodomain are able to translocate across the cytoplasmic membrane of mammalian cells. The translocation does not depend on cell endocytosis and it has been reported that translocation occurs at both 4° C. and 37° C. Homeodomain synthetic peptides made of D amino acids are also able to cross the cytoplasmic membrane. This finding would rule out the possibility that Antp is translocated through a receptor mediated mechanism. This property has been exploited to vehiculate small viral sequences into the cytoplasm of cultured cells as well as to elicit an MI-IC class I restricted cytotoxic immune response against the nucleoprotein of the influenza virus. However, to date, the homeodomain of Antp has only been used to transport small synthetic peptides.

Basic peptides, such as Drosophila Antennapedia or Tat of HIV-1, can promote cell internalization of linked peptides and peptidomimetic molecules. A truncated HIV-1 Tat protein basic domain rapidly translocates through the plasma membrane and accumulates in the cell nucleus. Non-natural basic peptides endowed with cell-penetrating properties have also been synthesized. These peptides are collectively called protein transduction domains (PTDs). Peptides, antisense oligonucleotides, and proteins conjugated to PTDs have been noted to internalize effectively, and their biological actions have been detected in several cell and animal models. This non-invasive approach for intracellular delivery of biologically active macromolecules is potentially a very powerful strategy, because intracellular protein targets can be attacked directly.

The high specificity and long active half-life of antibodies and their recombinant fragments make them excellent candidates for selective targeting agents. Single chain fragment variable (scFv) and monoclonal antibodies (mAbs) are capable of adopting a functional three-dimensional conformation joining together a VH and VL domain. The molecular mass of a standard IgG antibody is 150,000 Da and that of an scFv antibody is 30,000 Da, therefore it is potentially feasible to internalize whole IgG as well as a smaller scFv molecule. Single-chain mAb expression within the cell can be effectively obtained using recombinant DNA transfection techniques however, low general accessibility of target cells to DNA constructs together with lack of pharmacological modulation of antibody levels poses limitations from the perspective of therapeutic applications.

### SUMMARY OF THE INVENTION

The present invention is based on the seminal discovery that an antibody or fragment thereof combined with Antennapedia, is an effective therapeutic agent. The invention describes the construction of antibody or antibody fragment recombinantly fused or chemically conjugated with Antennapedia at its carboxyl or its amino terminus (a “cargo-carrier” construct). Surprisingly, Antp is able to transport antibodies which are large complex molecules.

Antennapedia-antibody or antibody fragment conjugates or constructs capable of penetrating cell membranes dramatically broaden the potential for innovative therapeutic agents. Internalization of a fluoresceinated antibody-Antp construct was observed in intact human cultured cells with confocal microscopy (see Examples). After a few hours of incubation in culture medium, fluorescence intensity was determined in individual cells, both for cytoplasmic and nuclear compartments. Concentration levels of the construct, relative to the extracellular culture medium concentration, were substantially higher in the cytoplasm, the nucleus, and the nucleoli. To silence gene functions by introducing recombinant vectors or proteins in cells has been a major goal in cell biology, originally achieved by means of a variety of invasive techniques such as microinjection, red cell ghost fusion, or electroporation, The present invention provides nucleic acid constructs and protein conjugates for delivery to cells.

Fusing PTDs to IgG and scFv antibodies as described herein allows the creation of a “cell-permeable” antibody, capable of effectively inhibiting the function of intracellular targets. These features make the invention molecules more effective from a therapeutic perspective.

The homeodomain of Antp can be used to translocate antibodies, including fragments thereof (e.g., scAb). One of the key advantages of the present invention is that the Antp homeodomain can be used to translocate functional and regulatory antibodies to cells.

### BRIEF DESCRIPTION OF THE DRAWINGS

FIG. 1 shows transport of fluoresceinated antibodies to cells. FIG. 1A—Human Embryonic Kidney (293) Cells; FIG. 1B—Human Prostate Cancer (PC3) Cells.

### DETAILED DESCRIPTION OF THE INVENTION

The present invention is based on the finding that the homeodomain of Antp can be used to transport an antibody or fragment thereof into a cell more effectively than prior delivery vehicles. Such fusion proteins or conjugates provide effective regulatory or therapeutic compositions.

“Antibody” as used herein includes immunoglobulins which are the product of B cells and variants thereof as well as the T cell receptor (TcR) which is the product of T cells and variants thereof. An immunoglobulin is a protein comprising one or more polypeptides substantially encoded by the immunoglobulin kappa and lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Also subclasses of the heavy chain are known. For example, IgG heavy chains in humans can be any of IgG1, IgG2, IgG3 and IgG4 subclass.

Antibodies of the invention can be chimeric, humanized, or fully human or murine antibodies, and antigen-binding portions thereof, for example. Various forms of the antibody are contemplated herein. For example, a monoclonal antibody of the invention may comprise or consist of an intact antibody (i.e., full-length, having an intact Fc region), a substantially intact antibody, an antigen-binding portion thereof (e.g., a Fab, Fab′, F(ab′)₂) or a single chain Fv fragment. It is understood that all such forms of the antibodies are encompassed herein and throughout within the term “antibody.” Furthermore, an antibody of the invention may be labeled with a detectable label. Furthermore, antibodies of the invention are contemplated to be of monoclonal origin even though they may differ in glycosylation pattern.

While an Antp-Ab conjugate is an effective therapeutic, optionally, the fusion polypeptide including Antp and an antibody or fragment thereof, can further include small molecule organic compounds of 5,000 daltons or less such as drugs, proteins, peptides, peptidomimetics, glycoproteins, proteoglycans, lipids glycolipids, phospholipids, lipopolysaccharide, nucleic acids, proteoglycans, carbohydrates, and the like. Additional targeting agents may include well known therapeutic compounds including anti-neoplastic agents. Anti-neoplastic targeting agents may include paclitaxel, daunorubicin, doxorubicin, caminomycin, 4′-epiadriamycin, 4-demethoxy-daunomycin, 11-deoxydaunorubicin, 13-deoxydaunorubicin, adriamycin-14-benzoate, adriamycin-14-octanoate, adriamycin-14-naphthalen-eacetate, vinblastine, vincristine, mitomycin C, N-methyl mitomycin C, bleomycin A₂, dideazatetrahydrofolic acid, aminopterin, methotrexate, cholchicine and cisplatin, and the like. Anti-microbial agents include aminoglycosides including gentamicin, antiviral compounds such as rifampicin, 3′-azido-3′-deoxythymidine (AZT) and acyclovir, antifungal agents such as azoles including fluconazole, plyre macrolides such as amphotericin B, and candicidin, anti-parasitic compounds such as antimonials, and the like. Hormone targeting agents include toxins such as diphtheria toxin, cytokines such as CSF, GSF, GMCSF, TNF, erythropoietin, immunomodulators or cytokines such as the interferons or interleukins, a neuropeptide, reproductive hormone such as HGH, FSH, or LH, thyroid hormone, neurotransmitters such as acetylcholine, and hormone receptors such as the estrogen receptor.

An Antp-antibody targeting agent, including any linking moiety necessary for covalently linking Antp with the antibody or with a targeting agent to an amino acid residue of the antibody, may be at least about 1-300 daltons in size, and may be at least about 400, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500 or even 5,000 daltons in size, with even larger sizes possible.

In another embodiment, the invention provides a pharmaceutical composition comprising an Antp-antibody conjugate of the invention. The pharmaceutical composition of the invention may further comprise a pharmaceutically acceptable carrier. In the pharmaceutical composition, the antibody conjugate of the invention is the active ingredient. Preferably the pharmaceutical composition comprises a homogeneous or substantially homogeneous population of an antibody of the invention. The composition for therapeutic use is sterile and may be lyophilized, optionally supplied with an appropriate diluent.

The antibody conjugate of the invention may comprise a natural or synthetic homeodomain of antennapedia. The homeodomain of the Antp gene obtainable from Drosophila is shown in SEQ ID NO:1. (SEQ ID NO:1 Arg Lys Arg Gly Arg Gln Thr Tyr Thr Arg Tyr Gln Thr Leu Glu Leu Glu Lys Glu Phe His Phe Asn Arg Tyr Leu Thr Arg Arg Ark Arg Ile Glu Ile Ala His Ala Leu Cys Leu Thr Glu Arg Gln Ile Lys Ile Trp Phe Gln Asn Arg Arg Met Lys Trp Lys Lys Glu Asn). Sequences homologous to this homeodomain have been isolated from other organisms, including vertebrates, mammals and humans, and these are included in the present invention. The homeodomain may be prepared using standard techniques such as cloning using the procedure described in Joliet et al. (1991) Antennapedia homeobox peptide regulates neural morphogenesis. Proc. Natl. Acad. Sci. 88:1864-1868. As previously indicated, differences in the sequences of such multicellular organisms are generally conservative in nature. However, this may not necessarily be the case and other such sequences are included in the present invention, and for example where the sequence identity is about 50% or more, e.g., 60%, 70%, 80% or 90%, with the sequence obtainable from Drosophila. Sequence identity may be determined using such commercially available programmes as GAP.

In addition synthetic or other variants may be used provided that they retain the ability to translocate the membrane. Synthetic or other variants may differ from the naturally-occurring proteins by substitution, particularly conservative substitution. By conservative amino acid changes is meant replacing an amino acid from one of the amino acid groups, namely hydrophobic, polar, acidic or basic, with an amino acid from within the same group. An example of such a change is the replacement of valine by methionine and vice versa. Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded fusion protein using the DNA sequence encoding the antibody of choice. Antp is ligated at either 5′ or 3′ terminus of the antibody sequence. These methods are only illustrative of the numerous standard techniques known in the art for manipulation of DNA sequences and other known techniques may also be used.

The ability of a naturally occurring or synthetic sequence to translocate the membrane may be tested by routine methods known in the art and illustrated in the accompanying examples. Some variants of the homeodomain which retain the ability to translocate the membrane have been reported in the art and these are included in the scope of the present invention, together with any which become available. For example, EP-B-0 485 578 discloses homeopeptides comprising the helix 3 sequence of Antp, and these are incorporated herein by reference. WO97/12912 discloses the actual sequence of the helix 3 of Antp, and variants thereof. Other variants are disclosed in for example, Gehring W (1987) Homeo Boxes in the Study of Development. Science 236 1245-1252 discloses a homeodomain of 62 amino acids, i.e. with glu at position 0 and lys at position 61. Bloch-Gallego E at al (1993) Antennapedia Homeobox Peptide Enhances Growth and Branching of Embryonic Chicken Motoneurons In Vitro. The Journal of Cell Biology 120(2) 485-492 discloses a mutant called pAntp40P2 that was still able to translocate through the motoneuron membrane and to reach the nucleus. In this mutant the leucine and threonine residues in positions 40 and 41 were replaced by two proline residues. Le Roux et al (1993) Neurotropic activity of the Antennapedia homeodomain depends on its specific DNA-binding properties. Proc. Natl. Acad. Sci. 90 9120-9124 discloses two mutants pAntp 50A and pAntp 40P2 which retain the ability to translocate through the neuronal membrane. Schutze-Redelmeier M-P et al (1996) supra disclose that a 16 amino acid C-terminal (third helix) segment has been used to address oligonucleotides and oligopeptides to the cytoplasm and nuclei of cells in culture.

When the Antp and antibody regions are conjugated via a linker, the linker can be a cleavable linker region. Preferably, the cleavable linker region is a protease cleavable linker, although other linkers, cleavable for example by small molecules, may be used. These include Met-X sites, cleavable by cyanogen bromide, Asn-Gly, cleavable by hydroxylamine, Asp-Pro, cleavable by weak acid and Trp-X cleavable by, inter alia, NBS-skatole. Protease cleavage sites are preferred due to the milder cleavage conditions necessary and are found in, for example, factor Xa, thrombin and collagenase. Any of these may be used. The precise sequences are available in the art and the skilled person will have no difficulty in selecting a suitable cleavage site. By way of example, the protease cleavage region targeted by Factor Xa is I E G R. The protease cleavage region targeted by Enterokinase is D D D D K. The protease cleavage region targeted by Thrombin is L V P R G. Preferably the cleavable linker region is one which is targeted by endocellular proteases.

Antp may be used to transport into cancer cells antibody molecules that regulate transcription factors and are able to restore cell cycle control or induce differentiation. For example, it is understood that many cancer cells would undergo apoptosis if a functional p53 molecule is upregulated. The present invention may be used to deliver such antibody products to directly or indirectly regulate genes or proteins.

The Antibody-Antp molecules of the invention are useful for antibacterial and antiviral measures. For example, Antp may be used to transport antibodies in the cytoplasm of viral or bacterial or other pathogen-infected cells, antibodies which interfere with a crucial step of bacterial and viral replication.

The antibody conjugates of the invention are useful for the treatment of diseases and disorders, for example but not limited to: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

The term cancer as used herein refers to proliferative diseases, such as lymphomas, lymphocytic leukemias, lung cancer, non small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewing's sarcoma, including refractory versions of any of the above cancers, or a combination of one or more of the above cancers.

Antp-Antibody conjugates can be used for a variety of activities and diseases, including for example, macrophage inhibitory and/or T cell inhibitory activity and thus, anti-inflammatory activity; anti-immune activity, e.g., inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g., retinitis or cystoid macular oedema, sympathetic ophthalmia, scieritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g., following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g., due to infection with a viral carrier, or inflammation associated with AIDS, to suppress or inhibit a humoral and/or cellular immune response, to treat or ameliorate monocyte or leukocyte proliferative diseases, e.g., leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

As used herein, “pharmaceutical carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion

Where appropriate, the pharmaceutical compositions can be administered by any one or more of inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner. The delivery of an Antp-antibody conjugate of the invention may be used alone or in combination with other treatments or components of the treatment.

In some embodiments, the Antp-antibody conjugate is used alone or a pharmaceutical composition comprising the Antp-antibody conjugate and a pharmaceutically acceptable carrier or excipient may be used in a method of treating a subject, comprising administering a therapeutically effective amount of the Antp-antibody conjugate to a subject.

In preferred embodiments, the subject is a mammal. Exemplary mammals include human, pig, sheep, goat, horse, mouse, dog, cat, cow, etc. Diseases that may be treated with the Antp-antibody conjugate include cancer, such as cancer of the skin, head and neck, lung, breast, prostate, ovaries, endometrium, cervix, colon, rectum, bladder, brain, stomach, pancreas or lymphatic system may be treated. Patients suffering from B- or T-cell cancer, non-Hodgkin's lymphoma, Hodgkin's disease, lymphatic or myeloid leukemias, multiple myeloma, sarcoma and melanoma may be treated by administration of a therapeutic amount of the antibody-drug conjugate of the present invention.

The Antp-antibody conjugate may be administered intravenously, intra-peritoneally, intra-arterially, intra-thecally, intra-vesically, or intratumorally. The conjugate may be given as a bolus or as an infusion on a repeat and/or a cyclical basis. The infusion may be repeated for one or more times depending on the dose of drug and tolerability of the conjugate in terms of side effects and is determined by the managing physician. One of ordinary skill will appreciate that effective amounts of the antibody-drug conjugate can be determined empirically. The agents can be administered to a subject, in need of treatment of cancer, as pharmaceutical compositions in combination with one or more pharmaceutically acceptable excipients. It will be understood that, when administered to a human patient, the total daily usage of the agents or composition will be decided by the attending physician within the scope of sound medical judgment. The specific therapeutically effective dose level for any particular patient will depend upon a variety of factors: the type and degree of the cellular response to be achieved; activity of the specific Antp-antibody conjugate or composition employed; the specific Antp-antibody conjugate or composition employed; the age, body weight, general health, sex and diet of the patient; the time of administration, route of administration, and rate of excretion of the agent; the duration of the treatment; drugs used in combination or coincidental with the specific agent; and like factors well known in the medical arts. For example, it is well within the skill of the art to start doses of the agents at levels lower than those required to achieve the desired therapeutic effect and to-gradually increase the dosages until the desired effect is achieved.

In one embodiment, the Antp-antibody conjugate is administered preceded by, concomitantly with, or subsequent to other standard therapies including radiotherapy, surgery or chemotherapy.

In one embodiment, two or more conjugates of an antibody and Antp are administered which conjugates affect different targets in the same diseased cells. In yet another embodiment, a conjugate of an antibody and an anthracycline drug is administered, preceded by, concomitantly with, or subsequent to another antibody-based treatment. This additional antibody-based treatment may include the administration of two or more antibody-based treatments, to include naked therapy, where the antibody is administered alone or in combination with another therapeutic-agent that is administered either conjugated or unconjugated to the antibody. The conjugation may utilize the presently disclosed linker or another type linker. When two antibody-based treatments are administered, these treatment are such that whichever antibody is administered second targets a different antigen or a different epitope on the same antigen on diseased cells. The second antibody could also be conjugated with another (different) drug or with a therapeutic isotope, thus providing an antibody-based combination therapy. It is also appreciated that this therapy can be combined, with administration before, simultaneously, or after with cytokines that either enhance the antitumor effects or prevent or mitigate the side effects of the therapeutic conjugates.

Each of the above identified methods of treatment may additionally include the administration of one or more immunomodulators. These immunomodulators may be selected from the group consisting of interferons, cytokines, stem cell growth factors, colony-stimulating factors, lymphotoxins and other hematopoietic factors. The interferon is preferably alpha-interferon, beta-interferon or gamma-interferon and the hematopoietic factors may be selected from the group consisting of erythropoietin, thrombopoietin, interleukins (ILs), colony stimulating factors (CSF), granulocyte macrophage-colony stimulating factor (GM-CSF) and G-CSF. The interleukin may be selected from the group consisting of IL-1, IL-2, IL-3, IL-6, IL-10, IL-12, IL-18, and IL-21. The immunomodulator or hematopoietic factor may administered before, during, or after immunoconjugate therapy. The immunomodulator is administered to enhance the effectiveness of the administered conjugate of the present invention.

The term “valent” as used within the current application denotes the presence of a specified number of binding sites in an antibody molecule. As such, the terms “bivalent”, “tetravalent”, and “hexavalent” denote the presence of two binding site, four binding sites, and six binding sites, respectively, in an antibody molecule. Bispecific antibodies according to the invention are at least “bivalent” and may be “trivalent” or “multivalent” (e.g., (“tetravalent” or “hexavalent”). Preferably the bispecific antibody according to the invention is bivalent, trivalent or tetravalent. In one embodiment said bispecific antibody is bivalent. In one embodiment said bispecific antibody is trivalent. In one embodiment said bispecific antibody is tetravalent.

The term “recombinant human antibody”, as used herein, is intended to include all human antibodies that are prepared, expressed, created or isolated by recombinant means, such as antibodies isolated from a host cell such as a NSO or CHO cell or from an animal (e.g., a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo. The “variable domain” (variable domain of a light chain (VL), variable region of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a .beta.-sheet conformation and the CDRs may form loops connecting the beta-sheet structure. The CDRs in each chain are held in their three-dimensional structure by the framework regions and form together with the CDRs from the other chain the antigen binding site.

The Antp-antibody according to the invention is typically produced by recombinant means. Thus, one aspect of the current invention is a nucleic acid encoding the antibody and Antp, or portion thereof (e.g., homeodomain) according to the invention and a further aspect is a host cell comprising the nucleic acid encoding an Antp-antibody according to the invention. Methods for recombinant production are widely known in the state of the art and comprise protein expression in prokaryotic and eukaryotic cells with subsequent isolation of the Antp-antibody and usually purification to a pharmaceutically acceptable purity. For the expression of the antibodies as aforementioned in a host cell, nucleic acids encoding Antp fused with the respective modified light and heavy chains are inserted into expression vectors by standard methods. Expression is performed in appropriate prokaryotic or eukaryotic host cells like CHO cells, NSO cells, SP2/0 cells, HEK293 cells, COS cells, PER.C6 cells, yeast, or E. coli cells, and the antibody is recovered from the cells (supernatant or cells after lysis). General methods for recombinant production of antibodies are well-known in the state of the art and described. (For example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-160; Werner, R. G., Drug Res. 48 (1998) 870-880).

The Antp-antibodies are suitably separated from the culture medium by conventional immunoglobulin purification procedures such as, for example, antibody columns for Antp protein A-Sepharose, hydroxylapatite chromatography, gel electrophoresis, dialysis, or affinity chromatography. DNA and RNA encoding the antibodies is readily isolated and sequenced using conventional procedures. The hybridoma cells can serve as a source of such DNA and RNA. Once isolated, the DNA may be inserted into expression vectors, which are then transfected into host cells such as HEK 293 cells, CHO cells, or myeloma cells that do not otherwise produce immunoglobulin protein, to obtain the synthesis of recombinant antibodies in the host cells.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two or more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

The following examples are intended to illustrate but not limit the invention.

### Example 1

This example illustrates a fluorescence-labeled scFv-Antp construct. An anti-HIS antibody (cargo which is FITC tagged) with Antp (homeodomain peptide) was used for delivery. A very efficient internalization of the peptide/cargo construct was observed in intact cells kept in culture medium, utilizing confocal microscopy. Fluorescence intensity was determined in multiple spots of each individual cell examined.

In a recent study, penetration of a scFv-TAT against Bcl-XL in cultured cells was reported. These authors studied internalization in the presence of fixative agents (Cohen-Saidon, C., et al. (2003). Other authors have shown, however, that cell fixation, even in mild conditions, leads to the artifactual uptake of peptides (Richard et al., (2003), J. Biol. Chem. 278:585-590).

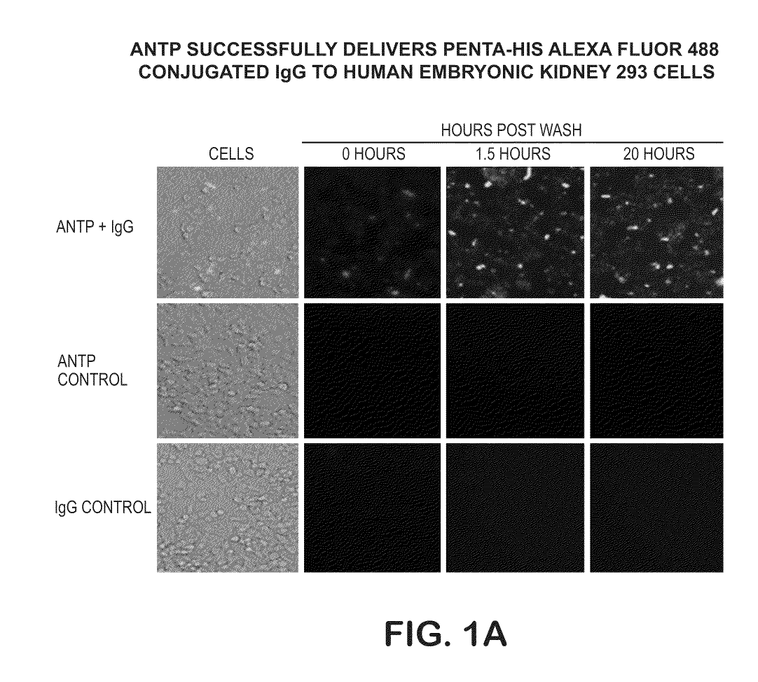

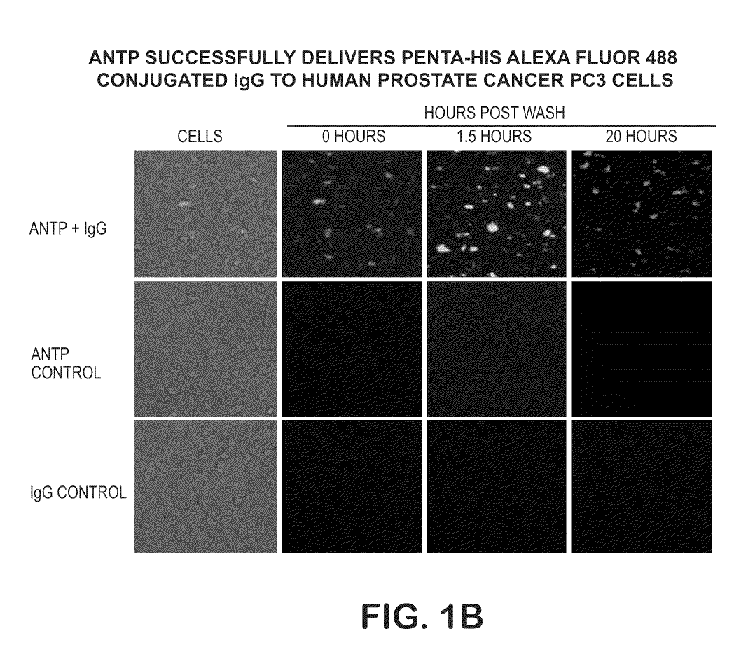

Aware of this artifact, the inventors studied the internalization of an scFv-Antp construct in unfixed living cells. Local fluorescence intensity of regions of interest was obtained, with the overt advantage of the reduction of background information away from the focal plane and the possibility of collecting serial sections. Pharmacologically attainable concentrations in a micromolar range for peptides or antibodies were used. Experiments performed demonstrate that internalization was achieved (see FIGS. 1A and 1B).

Experience with IgG and scFv antibodies showed that Kd values toward the target in a 10⁻⁸ to 10⁻¹⁰ M range can be often achieved (Hanes, J., Schaffitzel, C., Knappik, A., and Pluckthun, A. (2000) Nat. Biotechnol. 18, 1287-1292) and can be very specific and selective in terms of their interaction with the target. It was therefore especially interesting to have obtained very efficient internalization. These experiments show that Antp-Antibody molecules are endowed with a higher intracellular stability and improved pharmacodynamic and pharmacokinetic properties.

Finding good inhibitors of signaling proteins potentially important in sustaining a malignant neohomeostasis is crucial for modern cancer therapy. Monoclonal antibodies targeting membrane signaling proteins (of a dominant oncoprotein type, for instance, epidermal growth factor receptor family proteins, CD20 protein, and others) have had common and steadily expanding use in modern antineoplastic therapy.

The antibody-Antp nucleic acid constructs can be delivered to a cell using a vector. As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells. As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

Nucleotide sequences can be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Within certain embodiments, polynucleotides may be formulated so as to permit additional ease of entry into a cell of a mammal, and to permit expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art. Some embodiments of the invention have been described herein with Antp which inherently is a cell penetrating peptide/translocation peptide for entry into a cell.

Other formulations for therapeutic purposes include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Monoclonal antibodies against intracellular targets have been used extensively in basic research to silence gene functions in cells, by means of a number of techniques such as microinjection, red cell ghost fusion, or electroporation. This is, however, an approach suitable from the perspective of acquiring new basic biological knowledge, but not from a pharmacological/therapeutic perspective, for which reversible and dosage-modulated effects and the potential capability of entering practically every cell are crucial pharmacological requirements.

Fusing and/or constructing PTDs such as Antp to scFv and intact antibodies allows us to create a cell-permeable antibody, capable of effectively inhibiting the function of antigenic or sequence specific targets in cells.

Although the invention has been described with reference to the above example, it will be understood that modifications and variations are encompassed within the spirit and scope of the invention. Accordingly, the invention is limited only by the following claims.

What is claimed is:

1. A fusion protein comprising an antibody or fragment thereof fused or chemically conjugated to Antp or a fragment thereof.

2. The antibody of claim 1, wherein the antibody fragment is an scFv.

3. The antibody of claim 1, wherein the Antp is fused or conjugated at its amino or carboxyl terminus.

4. The antibody of claim 1, wherein the antibody is fluorescently labeled.

5. A nucleic acid sequence encoding the fusion protein of claim 1.

6. A host cell comprising the nucleic acid sequence of claim 5.

7. A method of treatment comprising administering to a cell a protein of claim 1 or nucleic acid sequence of claim 5 to the cell.

8. The method of claim 7, wherein the cell is a cancer cell.

9. The method of claim 7, wherein the cell is a mammal.

10. The method of claim 9, wherein the mammal is a human.
